(19) United States
(12) Patent Application Publication
(10) Pub. No.: US 2005/0256392 A1
(43) Pub. Date: Nov. 17, 2005

(54) Systems and methods for remote body imaging evaluation

(76) Inventors: Yvedt Matory, (US); Randall Kennedy, Dedham, MA (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/126,529

(22) Filed: May 10, 2005

Related U.S. Application Data
(60) Provisional application No. 60/570,308, filed on May 12, 2004.

(51) Int. Cl. A61B 5/05

(52) U.S. Cl. 600/407

(57) ABSTRACT

The disclosure describes techniques for use in a remote patient care system such as a remote patient care system that connects patients and health care professionals over a network using video conferencing.

### RELATED U.S. APPLICATION(S)

The present application claims priority to U.S. Provisional Application Ser. No. 60/570,308, filed May 12, 2004, which application is hereby incorporated herein by reference.

### TECHNICAL FIELD

The present invention is directed to body imaging techniques, and more particularly to remote body imaging protocol and remote evaluation of those images.

### BACKGROUND

Healthcare expenditures continue to represent the single largest sector of the U.S. economy, with over $1 trillion, or 14 percent, of the gross domestic product spent in 2000. Healthcare costs are at the highest level in two decades with no relief in sight. Hospitals and insurance providers are facing severe budget constraints due to shrinking reimbursements and higher costs of care.

Despite significant advances in healthcare delivery, the primary driver of controllable healthcare costs remains hospital stays. Latest figures from 1998 show that over 33% of total U.S. healthcare expenditures were a result of hospitalization (Health Affairs). Despite the strain of extended stays on care-givers, protracted time in a hospital does not necessarily translate into better quality-of-care for patients. Hospitalization can lead to significant potential medical complications, for instance, increase risks of infection, medication error, patient depression, which can further increase healthcare costs. Hospital acquired infections cost over $2 billion annually, according to a survey conducted from 1986 to 1998 by the National Nosocomial Infections Surveillance System of the CDC (eMedicine Journal). In addition, hospitalization-related medical errors lead to 98,000 deaths annually (Institute of Medicine). Prolong hospitalization can also exacerbate “bed-shortages” experienced in many hospitals.

There has been a significant drive to decrease the length of hospitalization and to develop ways of taking care of patients at home. Hospitals and insurance providers have, over the past decade, decreased length of stay through a variety of efforts, but have reached the point of diminishing returns. Specifically, traditional discharge planning procedures can often fail to provide adequate care to a patient after discharge. Often patients are left to manage their own dressings, monitor drainage, and adjust their own pain medication intake within the bounds of prescribed prescriptions. While, in some cases, a nurse will visit a patient at home to evaluate incisions, drainage, and vital signs, such visits may be abbreviated and far between.

Additionally, a patient must often coordinate his/her own care with many different providers. For example, a patient must often schedule follow-on visits with medical specialists, e.g., radiation oncologist, surgeon, and/or other specialists. Furthermore, a patient must, at times, coordinate access to emotional and psychological services, such as volunteer support, recovery aid, situational social workers, and psychiatric services supporting quality of life issues.

The establishment of more expanded care in the home can be further limited due to existing technology. Currently, technology exists to evaluate patients remotely in many ways. They include: remote heart rate, remote lung function, remote stethoscopes, remote weight scales, remote audio-visual communication. These functions, although may be useful in their own way, do not allow visualization of internal organs for specific determination of the medical problem. Whereas these measurements are indirect indicators of existing medical problems, imaging data can be more specific and allow for a more accurate evaluation and diagnosis.

### SUMMARY OF THE INVENTION

The present invention, in one embodiment, provides a system for remote body imaging that can be used in connection with a remote patient care system, such as a remote patient care system that connects patients and health care professionals over a network using video conferencing.

In accordance with one embodiment, the system includes an image capturing device and a display screen in communication with the image capturing device displaying, in real time, a captured image. The system also includes an orientation mechanism displayed on the screen against which the captured image may be aligned for subsequent identification of an area of concern on the body part. The system further includes data transmission means connected to a public network for transmitting, in real time, captured image data to a remote location for evaluation.

In accordance with another embodiment of the present invention, a method for remotely evaluating a body image is provided. The method includes, among other things, positioning an image capturing device proximate to a body part to be imaged. Next, a captured image of the body part may be displayed, in real time, on a display screen. Thereafter, the image captured may be aligned against an orientation mechanism displayed on the screen for subsequent identification of an area of concern on the body part. The image captured then can be transmitted, in real time, to a remotely located screen. The transmitted image subsequently can be viewed from the remotely located screen for evaluation purposes.

### BRIEF DESCRIPTION OF THE DRAWINGS

FIG. 1 is a diagram of a system for remote disease management.

FIG. 2 is a diagram of a graphical user interface presented to a care provider.

FIG. 3 is a diagram of a graphical user interface presented to a patient.

FIG. 4 is a flow-chart of a process for remote disease management.

FIG. 5 is a flow-chart of a process for remote body imaging evaluation.

FIG. 6 is a flow-chart of a process for adjusting a remote disease management process.

FIG. 7 is a diagram of a computer platform suitable for adjusting protocol criteria based on collected data.

### DETAILED DESCRIPTION OF SPECIFIC EMBODIMENTS

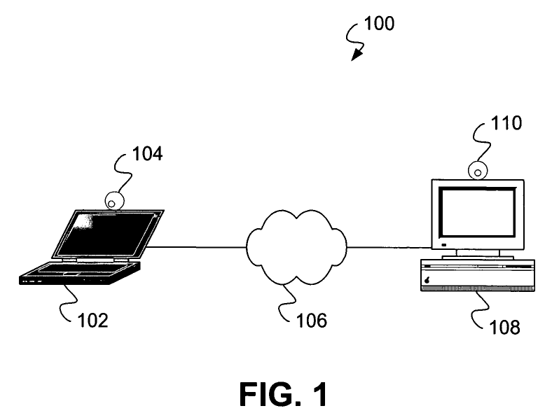

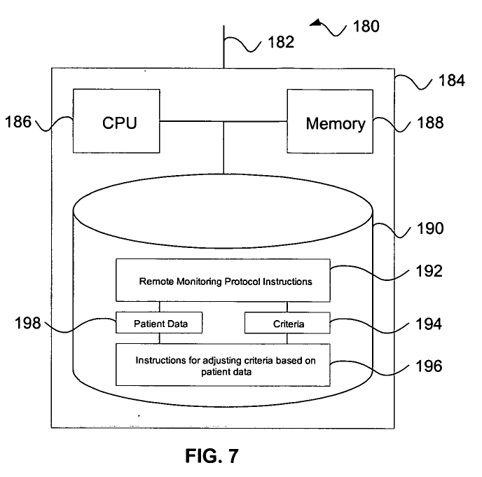

FIG. 1 shows a system 100 that enables health care professionals to remotely monitor and provide care to patients. As shown, the system 100 includes a patient's computer 102 and a health care provider's computer 108 that share data over a network 106, such as the Internet. While shown as a laptop 104, the patient's computer 102 may be a desktop model, Web TV, handheld device, wireless unit, and so forth. The system 100 may also include auxiliary computers such as an administrative computer (described in conjunction with FIG. 7).

Both patient and health care provider computers 102, 108 feature video cameras 104, 110 and microphones (not shown) for acquiring still-images, audio, and/or video data. An ultrasound imaging system may also be provided in connection with patient computer 102. The computers 102, 108 can communicate using network conferencing software such as Microsoft's NetMeeting or CUSeeMe. Instead of these off-the-shelf options, the computers 102, 108 may use dedicated conferencing/communication software developed for the system. Use of real-time conferencing enables health care professionals to provide patients with live interactive care without inconvenient travel to a hospital or extended time in a waiting room.

The system 100 offers an integrated approach to patient care and offers features that ensure proper treatment. For example, as explained below, the system 100 can dynamically adjust care parameters based on patient outcomes, satisfaction surveys, and other collected data. Additionally, as described below, the system 100 can provide a script for health care providers using the system 100 to maintain a high level of care.

The system 100 can enable hospitals to discharge patients earlier than traditionally contemplated while increasing the quality-of-care experienced by a patient. For example, patients can more quickly return to the personal comfort and reassurance of home. Additionally, unlike patients discharged after a lengthy hospital stay, patients using the system 100 enjoy continued access to hospital staff.

In addition to greater patient satisfaction and improved quality-of-care, the system 100 offers cost savings to many in the health care landscape. For example, by decreasing the use of costly in-patient and out-patient resources, hospitals reduce the financial obligations of insurers and hospital networks. Additionally, remote monitoring can greatly increase the productivity of health care professionals. For example, a nurse using the system can quickly monitor many patients without leaving their chair.

The system 100 uses a number of safeguards to ensure patient confidentiality while transmitting data over the public network 106. For example, the system 100 can use standard methods of encryption such as using Secure Sockets Layer (SSL) software. To further enhance security, the system 100 independently transmits and encrypts visual, audio, text, health care metrics (e.g., vital signs), and other information. The system 100 may also make use of passcodes to enhance security. The exchange of information complies with Health Insurance Portability Accountability Act (HIPAA) regulations.

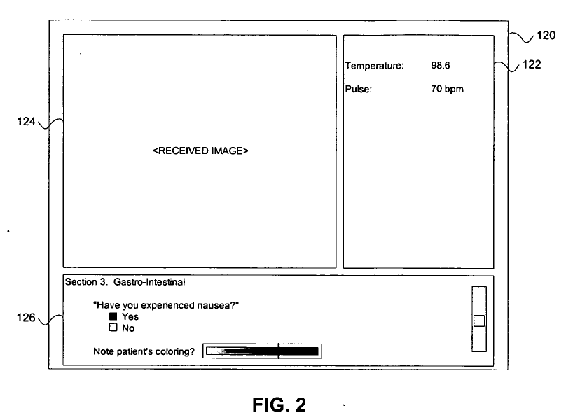

FIG. 2 shows an example of a user interface 120 presented to a health care professional during a remote care session. The interface 120 enables a professional to remotely assess patient status against care management guidelines for the patient's clinical condition. The user interface 120 includes a region 124 for viewing image/video data transmitted by the patient's computer. The user interface 120 can also present other data collected and transmitted by the patient computer. For example, the patient's computer may be equipped with sensors and other devices for collecting heart rate, blood pressure, glucose levels, spirometry, as well as ultrasound imaging devices, and so forth. The user interface 120 can dynamically update the display of these values.

The information presented by the user interface 120 enables a nurse to gauge a patient's condition, advise when a patient needs to be seen in the physician office, and alert the nurse to request other information or views of the patient. The user interface 120 may also provide controls (not shown) that enable the health professional to remotely control the patient's camera, for example, by changing its orientation and/or magnification.

As shown, the user interface 120 also presents a concurrently displayed script 126 region that provides guidance to a health care professional during a patient session. The script can remind the health care professional to ask certain questions, note particular aspects of a patient, and so forth. As shown, the script 126 can also receive data entry via familiar user interface control “widgets”, such as radio buttons, sliding scales, text boxes, and so forth. As the nurse responds to script 126 questions and prompts, the script 126 instructions can store the responses and determine the next questions/statements to present.

The particular script 126 selected for use during a remote session may depend on the particular ailment, patient, duration since last visit, and other factors. Additionally, the script 126 may incorporate conditional logic that varies the questions/prompts presented based on the patients previous responses or other collected information. For example, if the health care computer receives vital sign data indicating a quickened pulse, the script 126 logic may cause a question to be presented asking whether the patient feels feint. Similarly, as shown, if a patient reports nausea, the script 126 may present a color slide bar for the health care professional to manipulate to match the patient's pallor. The script 126 may also, in programmed circumstances, direct the nurse to contact a physician, for example, by presenting a “button” for the nurse to select. Alternatively, the script 126 may automatically initiate physician contact, for example, by paging or sending an e-mail. The script 126 may be encoded in a variety of formats such as Java Applets stored at a particular URL (Universal Resource Locator).

The user interface 120 may present other information. For example, the interface 120 can graph collected data, such as, ultrasound images or a graph of lung function over time. Additionally, the user interface 120 may provide access (not shown) to reference material for the health care professional conducting the remote session. Further, the user interface 120 may provide links (not shown) to other hospital facilities, for example, to schedule a visit with another health care professional.

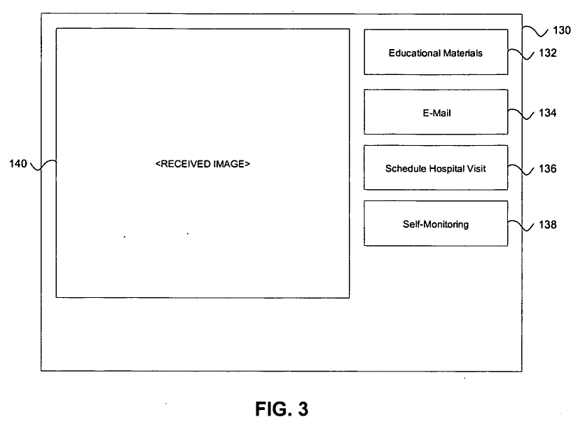

FIG. 3 shows an example of a user interface 130 presented to a patient. As shown, the interface 130 includes a region 140 for presenting images/video received from the health care computer. While not strictly necessary, presenting images of a health care provider can increase a patient's perception of personal attention.

As shown, the patient's user interface 130 also provides access to services that can be accessed even when a remote care session is not in progress. For example, the interface 130 provides access to personally tailored educational materials 132 that can let patients discover answers to common questions at their own pace. The interface 130 can also provide access to an e-mail 134 service that enables patients to e-mail information to a health care provider. For example, a patient can send an e-mail to a doctor or nurse that includes a still image or video of an operation site and the text of a question regarding the image(s). The user interface 130 can also provide access to other hospital systems, for example, to schedule appointments 136, check staff credentials, check prescriptions, and so forth.

The system may also enable a patient to interact with their own treatment plan off-line. For example, the patient's computer may receive computer instructions and/or data from a health care provider that can automatically provide features traditionally provided by human health care providers. For example, the instructions can provide video or text that guides a patient through a data acquisition process. For instance, the instructions may describe and depict a series of steps needed to take a body image using the ultrasound equipment connected to the patient's computer. The instructions may respond to a provided schedule or acquired information (e.g., answers to additional questions, previous measurements, and a doctor's treatment plan encoded in the instructions or data) by suggesting a patient action. In certain instances, the instructions may automatically initiate contact (e.g., page or e-mail) with hospital personnel or instruct the patient to do so.

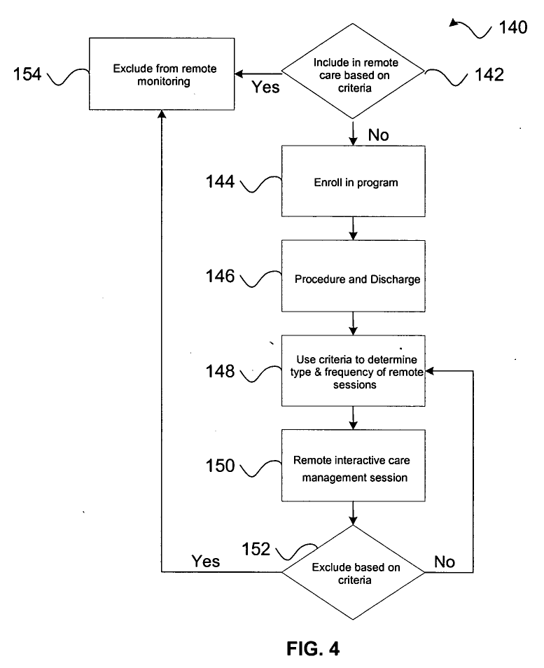

FIG. 4 illustrates a protocol 140 for use with the remote care management system. The protocol 140 helps ensure that remote care does not replace in-person care needed by some patients. The protocol 140 also helps tailor the remote care process to the needs of a particular patient. For example, the protocol 140 can adjust the frequency of remote sessions based on patient characteristics.

The protocol 140 shown is merely exemplary and may vary at different sites and for different purposes. For illustration purposes, this application describes the protocol 140 within the context of a remote monitoring/evaluation protocol 140.

After a normally required hospital admission for a particular medical procedure, patient characteristics are compared 142 to criteria to determine whether remote monitoring/evaluation is appropriate for the patient. Such criteria may include criteria requiring a patient to live within a certain threshold driving distance to a hospital, have a telephone line, have some self-reported or observed familiarity with computers, reside in a home within someone able to assist with physical care, have no co-morbid diseases, a physician referral, and so forth. These criteria are merely examples. Again, these criteria may be removed or altered and others added based on patient satisfaction, outcomes, financial impact, and so forth.

The protocol 140 enrolls 144 patients that meet these criteria and that agree to participate. Enrolled patients receive a computer and instructions, for example, when they come to the hospital for pre-procedure testing. Patients may meet with the nursing staff that will be giving them the post-procedure computer visits. To confirm that they understand the use of the computer, patients receive a trial computer visit prior to their surgery.

After the procedure and discharge 146, patients may receive scheduled remote interactive care management visits 150. For example, the patient may receive an e-mailed schedule identifying times to turn on their computers.

During the remote care management visits 150, nurses, for instance, may use the system to remotely interact with patients and respond in real time. For example, nurses can ask the patients specific questions, examine particular conditions, review care procedures, and so forth, for example, in accordance with the script described in conjunction with FIG. 3. For instance, in response to a patient's comment that a certain area on the body is in pain, a script may suggest asking the patient to initiate an ultrasound image capturing event of the area in pain and thereafter to describe the severity of the pain and the exact location of the pain. In addition to receiving data from the attached equipment, the nurse can note the patient's appearance as presented by the received video image. Again, the data collected during the interactive visit is stored for subsequent analysis and, potentially, adjustment of protocol 140 criteria.

Enrollment does not limit patient access to more traditional care. For example, patients may call a tele-monitoring nurse or their doctor at any time, request a home visit, and/or schedule an appointment at a hospital. Additionally, even where remote visits form a portion of a patients care, a protocol 140 may schedule both remote and in-person appointments. An in-person post-procedure appointment with a physician may typically be scheduled for 10-14 days after the procedure. Assuming a satisfactory outcome, the patient returns the computer, completes a satisfaction questionnaire, and the patient's participation in the protocol 140 ends.

Through-out the study, the protocol 140 determines 152 whether remote monitoring/evaluation continues to offer an effective method of patient care. Again, the protocol 140 may use different criteria to make this determination 152. For example, the protocol 140 may evaluate a patients vital signs for instability (e.g., a temperature greater than 100, blood pressure less than 90/60 or over 160/100, and/or a pulse greater than 110), evidence of wound bleeding (e.g., conspicuous hematoma or drainage output greater than 100 cc in the first four hours), and/or inadequate pain control as reported and noted by the remote nurse.

The protocol 140 also uses criteria to determine 148 the type and frequency of remote monitoring. For example, the protocol 140 may use patient answers, staff notations, and other collected data to determine a time for the next visit(s). For instance, a slowly recovering patient may be scheduled for a next appointment at an earlier date than a quickly recovering patient.

As described above, in addition to health-based factors, the criteria described above may incorporate resource management considerations. For example, enrollment criteria may depend on the number of nurses trained in use of the system or other resources.

In connection with one embodiment of the present invention, the system 100 may be equipped with certain components to permit, for instance, generation of body images. There are a range of body imaging techniques currently available. They include, among others, roentgenograms abbreviated Xrays, magnetic resonance imaging, abbreviated MRI, and ultrasound. As ultrasound is portable in comparison to these other imaging techniques, the present invention contemplates the use of ultrasound, a technique that uses sound waves, to generate the necessary body images. It should be noted that although ultrasound is contemplated, the system of the present invention may be adapted for use with any feasible imaging technology.

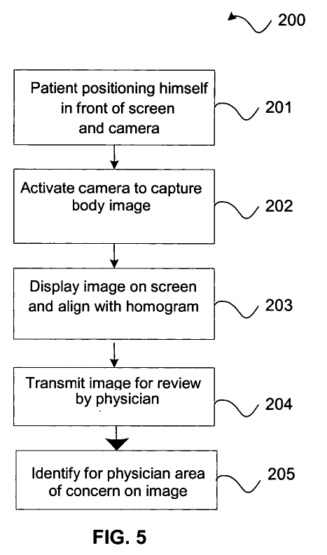

Looking now at FIG. 5, a protocol 200 for remote body imaging evaluation is provided in connection with the system 100 of the present invention. At a scheduled time, a patient may be asked to stand 201 before a camera, for instance camera 104 on a screen of computer 102 (see FIG. 1), so that an internal image of the body part with which the patient is most concerned may be generated by a camera, for example, x-ray, ultrasound, etc., connected to the screen. Once the patient is appropriately positioned before the screen, the patient may activate 202 the camera, for instance, by pushing a button connected directly or remotely to the camera to capture a body image, including an image of the area of concern. The captured body image may thereafter be displayed 203 on the screen of the computer 102 for the patient to review, so as to ensure that the area of concern has been captured. The picture may be generated on the screen as a two-dimensional or a three-dimensional image, and may be transmitted 204 in real time to a remotely located screen where it may be observed and evaluated by a tending physician. In one embodiment of the present invention, as it may be difficult to determine where on the body the image was taken, the image may be aligned 203 with a homogram, i.e., a picture of the body generated on the computer screen to appropriately orient and/or locate the image for the patient, and subsequently for the radiologist/physician who will be evaluating the image. The homogram, in one embodiment, may be two-dimensional or three-dimensional. The image and/or homogram may be transmitted in real time 204 by the system through, for example, a phone or cable line, or wirelessly, to a site at which the image can be displayed for review by the tending physician. Alternatively, the image may be stored and transmitted at a later time, should the patient is generating the picture at a time when the physician is not available. Using the homogram, the patient can identify 205 for the physician, the area of concern or the site of greatest discomfort. Based on the description by the patient and the image, the physician may make an appropriate evaluation.

In another embodiment of the present invention, rather than standing before a computer screen, the system may be provided with a probe (not shown) that can be used to generate an image on the computer screen. In this embodiment, the patient may use the probe to position it on the area or site of pain or concern, rather than having to strategically position himself in front of the screen and camera so that the area of concern can be captured. An image may thereafter be generated from the probe and displayed on the screen for the patient to review. The image may be aligned with a homogram to appropriately orient and/or locate the image for the patient and for the radiologist/physician who will be evaluating the image. The captured image may be transmitted by the system in real time through, for example, a phone or cable line, or wirelessly, to a site at which the image can be displayed for review by the tending physician. Alternatively, the image can be stored for later transmission. It should be noted that in this embodiment, as well as the immediately above embodiment, during real time transmission, a copy of the image can be stored. Using the homogram, the patient can identify for the physician, the area of concern or the site of greatest discomfort. Based on the description by the patient and the image, the physician may make an appropriate evaluation.

The system described above can used to evaluate many different conditions or for various medical purposes currently employed on an in-patient basis. By employing the use of various add-on components and/or peripherals, the system can be used to promote, for instance, early discharge by offering each preventive care education, monitoring adherence to self-care programs, and gauging patient response to treatment. The system can also be useful for remote wound care monitoring such as chronic leg ulcer management. Frequent monitoring and online reinforcement of self-care instructions can postpone or completely avoid the devastating affect of poorly attended skin trauma. The remote care system can also play an important role in treatment of, for example, diabetes and reducing in-patient days. For example, individuals with diabetes who have had an imbalance of serum glucose requiring inpatient management but who now have stable chemical results and stable cardio-respiratory status. Patient education, early preventive care, and consistent monitoring are important weapons in preventing many of the devastating vascular consequences of diabetes. The remote care system can also facilitate early discharge for stable maternity patients and offer convenient home care for infants and mothers during the post-partum period.

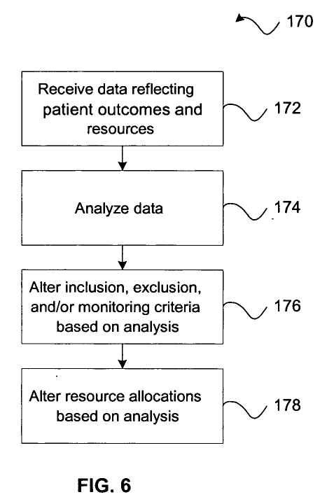

Referring to FIG. 6, the system continually monitors and reacts to the quality and cost of care received by remotely monitored patients. For example, the system may store and statistically analyze data describing patient outcomes, compliance, adverse events, and so forth. The system also monitors costs, charges, and reimbursement of the health care services as well as satisfaction surveys of physicians, payors, and vendors.

Based on this data, the system can modify criteria described above. For example, the system may automatically analyze the data to identify high correlations between criteria parameters and patient satisfaction, outcomes, or data reflecting a high cost. For example, if after time, statistical analysis of data indicates that patients over a certain age do not perform well with remote monitoring, the system may automatically raise the age criteria threshold for continued or initial participation. As another example, the system may identify certain patient conditions requiring more frequent remote sessions and correspondingly alter the protocol's remote session frequency for such patients.

The system may also aggregate data from different sites for comparison and subsequent modification of the protocol criteria. For example, the system may consider analyzing monthly and year-to-date results for aggregated member months, total inpatient costs, inpatient costs, total health provider admissions, admissions by inpatient facility, total inpatient days, inpatient days by health center provider, inpatient days by inpatient facility, and capitation revenue for inpatient care. Additionally, the system may consider average capitation revenue per member per month, average cost for inpatient care, number of admissions per 1000 members per year, number of patient days per 1000 members per year, average length of stay, average cost per day by facility, average cost per admission by facility, average length of stay by inpatient facility. The system may further evaluate on nursing time and activities. Again, based on analysis of this data, the system may automatically adjust the protocol, for example, by altering its criteria.

FIG. 7 depicts a computer 184 suitable for implementing aspects of the techniques described herein. As shown, the computer 184 includes a CPU 186 (Central Processing Unit), volatile memory 188, and non-volatile memory 190. The non-volatile memory 190 can store instructions 192 for implementing a protocol. The non-volatile memory 190 may also include instructions 196 for adjusting the protocol in response to collected data. Such instructions 196 may include instructions for statistically analyzing patient data 198 or other collected data. In the course of operation, the instructions 192, 196 are transferred from the non-volatile memory 190 to the volatile memory 188 and/or the CPU 186 for execution.

As shown, the computer 184 may also store protocol criteria and logic 194. The protocol logic 194 may be encoded using any of a variety of computer languages. The computer 184 may also store other information such as scripts (not shown) for use by health care professionals during a remote session and instructions that enable a user to access their treatment plan off-line.

As shown, the computer also features a network connection 182. As such, the features described above may be distributed across many different computers. For example, one computer may store patient data while another stores scripts for transmission to care taker computers.

The techniques described herein, however, are not limited to any particular hardware or software configuration. The techniques may be implemented in hardware or software, or a combination of the two. Preferably, the techniques are implemented in computer programs executing on programmable computers that each include a processor, a storage medium readable by the processor (including volatile and non-volatile memory and/or storage elements), at least one input device, and one or more output devices.

Each program is preferably implemented in high level procedural or object oriented programming language to communicate with a computer system. However, the programs can be implemented in assembly or machine language, if desired. In any case the language may be compiled or interpreted language.

Each such computer program is preferably stored on a storage medium or device (e.g., CD-ROM, hard disk, or magnetic disk) that is readable by a general or special purpose programmable computer for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains.

1. A system for use in remote body imaging evaluation, the system comprising:
an image capturing device;
a display screen in communication with the image capturing device for displaying, in real time, a captured image;
an orientation mechanism displayed on the screen against which the captured image can be aligned for subsequent identification of an area of concern on the body part; and
data transmission means connected to a public network for transmitting, in real time, the captured image to a remote location for evaluation.

2. A system as set forth in claim 1, wherein the orientation mechanism is a homogram.

3. A method of remotely evaluating a body image, the method comprising:
positioning an image capturing device proximate to a body part to be imaged;
displaying on a display screen, in real time, an image captured by the capturing device;
aligning the image captured against an orientation mechanism displayed on the screen for subsequent identification of an area of concern on the body part;
transmitting, in real time, the image captured to a remotely located screen; and
viewing, from the remotely located screen, the image transmitted for evaluation purposes.

4. A method as set forth in claim 3, wherein the step of aligning includes adjusting orientation of the image captured.
